(19) United States
(12) Patent Application Publication
(10) Pub. No.: US 2002/0065490 A1
(43) Pub. Date: May 30, 2002

(54) Syringe for medical purposes

(76) Inventors: Jochen Heinz, Vendersheim (DE); Michael Spallek, Ingelheim (DE)

Correspondence Address: Striker Striker & Stenby, 103 East Neck Road, Huntington, NY, 11743, US

(21) Appl. No.: 09/593,571

(22) Filed: Jun. 14, 2000

(30) Foreign Application Priority Data
Jun. 15, 1999 (DE) 199 27 201.8-41

(51) Int. Cl. A61M 5/24

(52) U.S. Cl. 604/205

(57) ABSTRACT

The syringe for transferring a medical substance from a container (10) provided with an elastomeric closure into an infusion container has a plastic syringe body (1). In order to avoid the danger of injury to a user by medical hollow needles previously used on syringes, a plastic hollow peg (9) is arranged at the front end of the plastic syringe body (1) instead of the typical sharp hollow needle. However the plastic hollow peg (9) is formed so that it can pierce the elastomeric closure (11) of the container (10). The hollow peg (9) is preferably in one piece with the syringe body (1). A transfer arrangement including a syringe (1), a preparation container (10) and a holder (10a) that holds the syringe on the preparation container is also described. The holder (10a) allows the syringe to be held in a storage position in which the hollow peg does not penetrate the elastomeric closure, but in which the syringe is closed, and in a position in which the contents of the preparation container can be drawn into the syringe.

### BACKGROUND OF THE INVENTION

1. Field of the Invention

The present invention relates to a syringe or injection device for medical purposes with a plastic syringe body, formed for transferring medical substances from containers, which are closed by an elastomeric closure, into an infusion container.

It also relates to a preferably pre-filled syringe. It is a matter of a filled syringe made from glass or plastic, which either has a metal needle glued to its head end or a conical part or section for receiving a medical needle.

2. Prior Art

All of the above-mentioned syringes or injection devices have the serious disadvantage that there is a danger of injuring the user with the needle. Thus the widest variety of expensive safety systems for needles was developed, in order to reduce the danger of injury from the needle.

The known filled syringes are well suited for direct injection into the patient. These syringes are poorly suited for drawing up a liquid medication from a standardized medication bottle provided with an elastomeric closure or a powdery medication from a standardized injector bottle.

Preparations, as they are used predominantly in medicine, pharmaceuticals and diagnostics and laboratory or analytical work, are primarily supplied commercially in the above-mentioned containers or are supplied in this form to the end consumer (user).

The preparations must be taken from the closed containers in a suitable way in each application, without impairing their functioning as much as possible. Frequently these preparations must be mixed with other substances prior to their use. For example, this is the case, when the preparations are present in solid form, i.e. when they are in the form of a powder or lyophilizate, which must be converted into a solution prior to use. Another example is the thinning of a liquid concentrated preparation.

Typically access to the container interior occurs by penetration of the elastomeric closure by means of a hollow needle and disposable syringe as “intermediate carrier”. When a liquid preparation is to be applied for example, the syringe is pulled out after penetration of the closing stopper of the container and is subsequently used to administer the preparation. When a mixture with another liquid substance should take place, the syringe first draws the other liquid substance up and then the stopper of the container is pierced and the syringe is emptied into it. After mixing the substances in the container the syringe draws up the mixed solution and subsequently applies it.

The steps of the method of application or use are similar when the active ingredient is present in solid form. First the injecting device or syringe draws up the solvent, the syringe pierces the stopper of the container holding the solid and after that is emptied into it. If necessary a mixing process can be performed with other substances. After that the syringe draws up the product solution to be applied.

If the syringe is a syringe of the above-descried type with a medical hollow needle, the medical hollow needle used can be easily damaged when it pierces the elastomeric closure of a container, which makes the exchange of the hollow needle routinely necessary. Injury to medical personnel or the user of the syringe can occur because of a required exchange of the hollow needle. Furthermore penetration of an elastomeric closure by use of a medical hollow needle causes the punching out of elastomer particles. There is thus a great danger that these elastomer particles are delivered together with the medication.

These disadvantages can be avoided in a known manner by closing the container with an integrated “transfer set”. The term “transfer set” means an additional device for conveying, i.e. transferring a preparation, directly, or after mixing with another substance, into a final container provided for the use, application, or directly into an applicator.

These transfer sets are especially suited for dissolving a powdery medication in the respective medication/injector bottles. They are not however suitable for transfer of the powdery medication directly into a rigid infusion bottle, since the necessary transfer pressure cannot be applied.

### SUMMARY OF THE INVENTION

It is an object of the present invention to provide a syringe for medical purposes of the above-described type with a plastic syringe body, formed for transferring a medical substance from a medical container, which is closed by an elastomeric closure, into an infusion container formed so that a reliable transfer of a powdery preparation found in the separate container into a rigid infusion bottle and also into a non-rigid infusion bag is guaranteed as simply as possible without using a medicinial syringe hollow needle.

According to the invention the front end of the syringe body has a hollow peg or pin made from a plastic material for penetration of the elastomeric closure.

Because of this feature an injector device or syringe for medical purposes, especially a pre-filled syringe, is provided, which can be used without a medical syringe hollow needle for penetration of the elastomeric closure.

The hollow peg, which can also be called a hollow spike, is sufficiently long and sharp so that it can penetrate the standard elastomeric closure of the infusion bottle made from plastic or glass and infusion bags, especially the standardized syringe stoppers, or standardized freeze-dry stoppers or standardized freeze-dried stoppers for infusion bottles.

The hollow peg guarantees that the danger of injury and injection of elastomer particles for the user is substantially less than in the case of medical hollow needles.

The hollow peg not only pierces the closure of the separate container in an easy manner, especially of medical/injection bottles, but also the closures of infusion bottles or infusion bags.

DE 77 02 734 U1 of course describes an arrangement for transferring medical substances from or into a container, which is closed by an elastomeric stopper, by means of a syringe, however this transfer happens with different means. While in the case of the invention the syringe body is provided directly with a hollow plastic peg on its front end as part of the syringe by means of which the elastomeric stopper is penetrated during each transfer process. In the known case a drawing and aerated hollow needle with a hollow plunge-cut piercing pin on its front end, separate from the syringe, is installed permanently in the container after piercing the stopper. This hollow needle has a filter for aeration of the container with germ-free air and a rubber insert for closing the passage in the hollow needle when it is not in use. The syringe itself in the known case only has a conventional cone-like injector head, with which the ends of the slotted rubber insert are spread in the case of a transfer, in order to form the opening for fluid transfer.

DE 77 02 734 U1 does not show the syringe according to the invention. It discloses a special closure insert introduced into the stopper of the container holding the medical liquid by means of the plunge-cut piercing pin, which allows it to perform repeated transfer of the medical liquid with a conventional syringe, without further piercing of the stopper.

EP 0 306 606 describes a transfer syringe with two hollow needles. One of the two hollow needles is used for piercing the stopper of the container and aeration during withdrawing of the medical liquid from the container. The other hollow needle is used for the “basic” injection for delivery of the medical liquid to the patient.

In both cases however typical injection hollow needles are used, which are made from a metal, i.e. the known “aspiration needle 01”. This known hollow needle is not a hollow peg or pin as in the case of the invention and also is not made of plastic material.

In the case of the invention however the use of medical hollow needles is avoided.

A conventional injector syringe is known from DE 21 64 363 A1, which has an injector needle made from plastic, but which is not a hollow peg in the sense of the invention. The injector syringe of this reference furthermore is not a transfer syringe in the sense of the present invention.

The syringe according to a further embodiment of the invention is formed so that the plastic hollow peg has a one-piece holder for penetration of a cone-shaped syringe head of the syringe body.

In this case the syringe is constructed in two pieces. The syringe body and hollow peg are made as separate parts. The hollow peg is then placed releasably on the syringe head with a holder.

According to another preferred embodiment of the invention however the syringe is preferably formed in one piece with the hollow peg, since the outlet end of the injector body is formed directly as a hollow peg.

The hollow peg can be formed in different ways according to the type of application, i.e. which type of closure should be penetrated. Thus according to a first embodiment it is conceivable that the hollow peg is a hollow cylinder, i.e. has a constant diameter over its entire length.

According to another embodiment the hollow peg can be conical and tapered, which increases the strength of the hollow peg at its base.

Also the penetrating end of the hollow peg can be formed in different ways. According to a first embodiment the hollow peg has an insertion end with a slant or a bevel over its entire diameter, whereby an eccentric tip is produced.

According to an additional embodiment of the invention the hollow peg for piercing is shaped like a cone and comes to a tip so that the piercing action is centered. The hollow peg can thus have only a single eccentrically arranged flow passage or it can alternatively have two symmetrically arranged flow passages. A greater flow results in the case of the latter embodiment.

In order to guarantee a sterile aeration of the application container, according to one embodiment of the invention a connector for attaching a sterile filter is formed on the body end of the hollow peg.

The invention also includes a pre-assembled syringe and container arrangement including a syringe for transferring a medical substance from a preparation container into an infusion container, which comprises a plastic syringe body having preferably two bulging projections spaced axially from each other on an exterior surface thereof and having a front end and a plastic hollow peg arranged on the front end of the plastic syringe body and the preparation container for holding the medical substance to be transferred to the infusion container. Preferably the preparation container has an elastomeric closure including a connector for engaging a front portion of the hollow peg to seal a connection thus formed between the preparation container and the syringe in a storage condition and the hollow peg includes means for penetrating the elastomeric closure to draw the medical substance from the preparation container. A holder for holding the syringe on the preparation container in a plurality of positions may also be provided. The holder includes means for engagement of the elastomeric closure with one bulging projection in the storage condition so that the syringe is closed by the connection with the connector but the hollow peg does not penetrate the elastomeric closure or alternatively with another of the bulging projections so that the elastomeric closure is penetrated by the hollow peg.

### BRIEF DESCRIPTION OF THE DRAWING

The objects, features and advantages of the invention will now be illustrated in more detail with the aid of the following description of the preferred embodiments, with reference to the accompanying figures in which:

FIG. 1 is a longitudinal cross-sectional view of a basic embodiment of a conventional syringe in a stored condition, which forms the basis for the syringe according to the invention,

FIG. 2 is a longitudinal cross-sectional view of the syringe of FIG. 1 ready for use with a cylindrical hollow peg according to the invention,

FIG. 3 is a cross-sectional action view of the syringe according to the invention shown in FIG. 2 in use with the pierced stopper of a medication bottle,

FIG. 4 is a longitudinal cross-sectional view through another syringe according to the invention that is similar to that of FIG. 2, but with a conical hollow peg,

FIG. 5 is a cross-sectional action view of the syringe shown in FIG. 4 in use, which is similar to FIG. 3,

FIG. 6 is a longitudinal cross-section view of another embodiment of the syringe according to the invention in a stored condition with the hollow peg in one piece with the syringe body,

FIG. 7 is a longitudinal cross-sectional action view of the syringe shown in FIG. 6 in use, which is analogous to FIG. 3,

FIG. 8 is a longitudinal cross-sectional view of another embodiment of a syringe according to the invention similar to the embodiment of FIG. 6, which is additionally provided with a sterile filter,

FIG. 9 is a longitudinal cross-sectional action view of the syringe shown in FIG. 6 in use,

FIG. 10 is a longitudinal cross-sectional view of an additional embodiment of a syringe according to the invention in which the hollow peg is formed in one piece with the syringe body; the hollow peg coming to a tip and having two symmetrically arranged flow passages,

FIG. 11 is a longitudinal cross-sectional action view of the syringe shown in FIG. 10 in use,

FIG. 12 is a longitudinal cross-sectional view of an additional embodiment of a syringe according to the invention in which the hollow peg is formed in one piece with the syringe body; the hollow peg coming to a tip and having an asymmetrically arranged flow passage,

FIG. 13 is a longitudinal cross-sectional view through the syringe shown in FIG. 6 provided with a holder during application with a medication bottle, and

FIG. 14 is a longitudinal cross-sectional view through a syringe according to FIG. 6, which has a standard hollow needle pushed on the hollow peg.

### DESCRIPTION OF THE PREFERRED EMBODIMENTS

FIG. 1 is conventional plastic syringe with a syringe body 1, which comprises a syringe cylinder 2 and a conical syringe head 3, which is closed by an elastomeric cap, a so-called tip-cap, 4. A piston stopper 5 with a piston rod 6 and a finger flange 7 on the syringe cylinder 2 completes the syringe body 1 of the conventional syringe.

FIG. 1 illustrates the basic structure of a known syringe. If the syringe is pre-filled and closed with the elastomeric cap, the piston stopper 5 rests at the lower end of the syringe cylinder 2 without adding the piston rod 6.

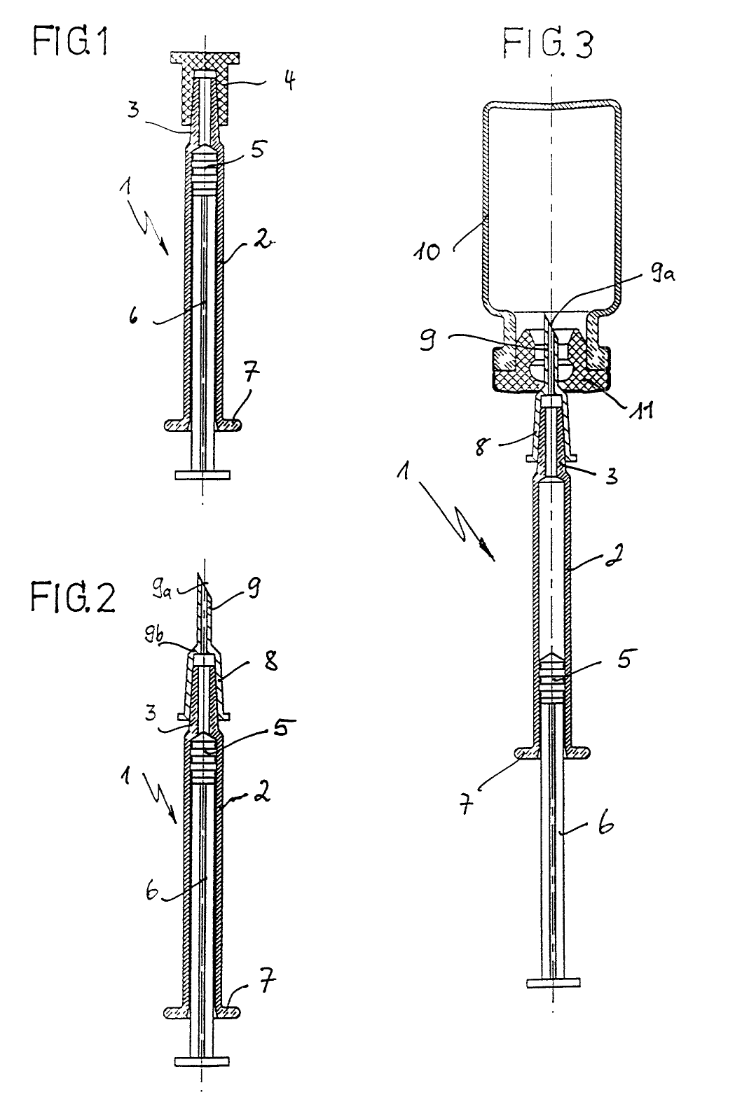

FIG. 2 shows the syringe according to the invention based on the basic structure according to FIG. 1 and ready for use. A one-piece hollow peg 9, the spike, is placed on the conical syringe head 3 by means of its complementarily-formed retaining section 8 after removing the elastomeric cap 4 from the known syringe of FIG. 1 to form the syringe according to the invention shown in FIG. 2. The retaining section 8 of the hollow peg 9 is made of plastic material and they are preferably injection-molded parts, like the syringe body.

The hollow peg 9 has a sharp edged bevel 9a on the front end of its interior flow passage, which makes the penetration of the elastomeric stopper easy, without a noteworthy injury danger.

FIG. 3 shows the syringe according to the invention in use in a medical bottle 10. The hollow peg 9 pierces the stopper 11 of the medical bottle 10, so that, according to the application, the contents of the syringe can be delivered to the medical bottle, e.g. for thinning of a solution contained in it or for dissolving a solid, e.g. freeze-dried, medical substance. After that the syringe can be drawn out in order to transfer its contents subsequently into an infusion container. This infusion container can be an infusion bottle made from glass or plastic with a standardized closure or an infusion bag with a conventional penetrable access opening, also called a port.

With the needle according to FIGS. 1 to 3 the retaining section 8 of the hollow peg 9 on the front end has a truncated-cone-shaped tapering portion 9b connected to the hollow peg 9, which has the same diameter over its entire length.

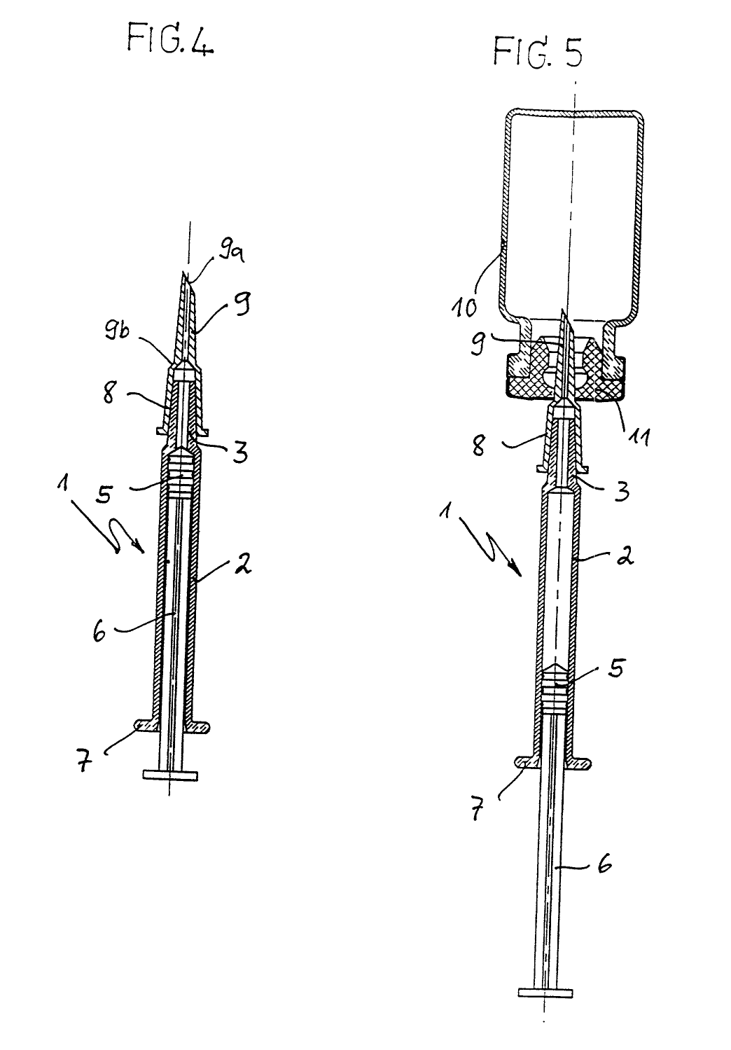

The truncated-cone-shaped portion 9b in the embodiment of the syringe according to the invention shown in FIGS. 4 and 5 is formed less strong than in the case of the embodiment of FIGS. 1 to 3. The hollow peg 9 in this embodiment is conically tapered to a point. Furthermore the hollow peg is longer than in the first embodiment. It can thus penetrate deeper into the container 10, as shown from FIG. 5.

Otherwise the embodiment of the syringe shown in FIGS. 4 and 5 corresponds to that shown in FIGS. 1 to 3.

In the two-piece embodiments of the injector or syringe shown in FIGS. 1 to 5 the hollow peg 9 with its retaining section 8 is formed as a separate part, which is placed on the syringe head 3.

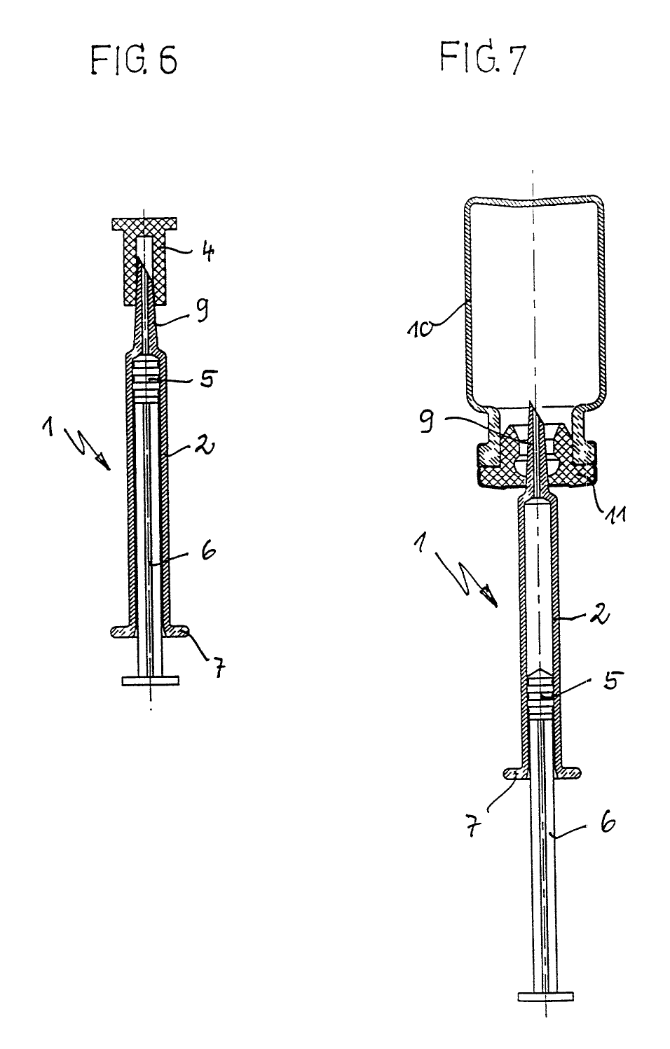

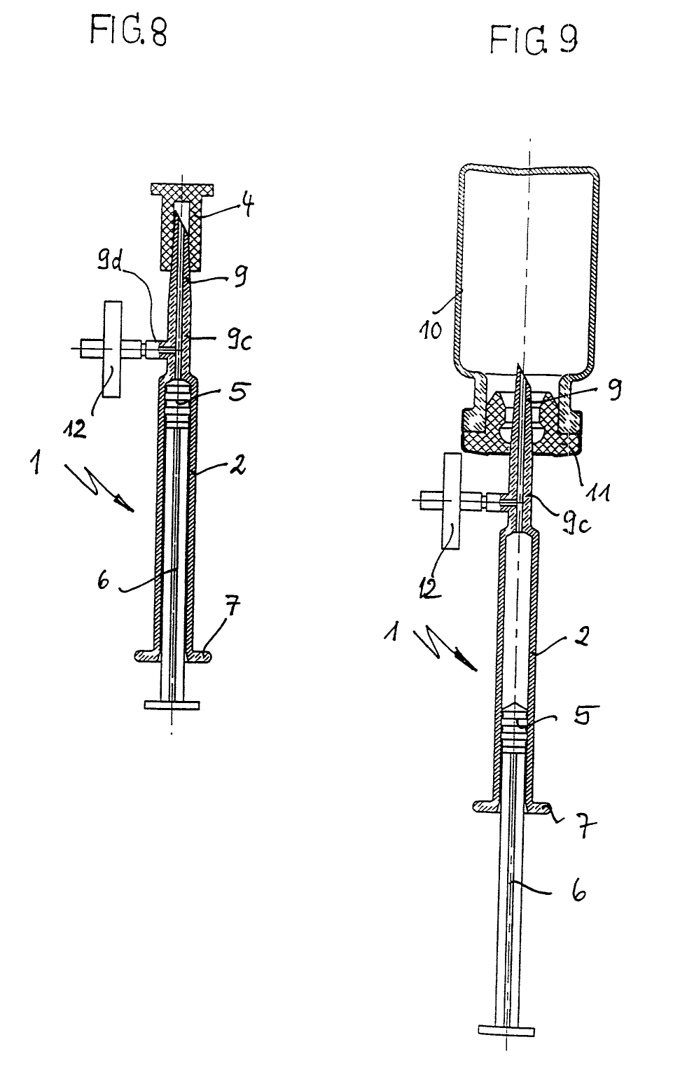

FIGS. 6 and 7 show a syringe with a front portion, which is directly formed as a hollow peg 9, which is in one-piece with the remaining portion of the syringe. In other words, the syringe head 3 of the embodiment of FIG. 1 is formed in a manner similar to the hollow peg 9 in the embodiment of FIGS. 6 and 7. The hollow peg 9 must be formed so as to be able to penetrate the closure of the container 10.

The hollow peg 9 in the embodiment of FIGS. 6 and 7 can have an equal diameter along its entire length as in the case of the embodiment of FIGS. 1 to 3. It can also, as illustrated however, be conical and tapering as in the case of the embodiment of FIGS. 4 and 5.

The embodiment of FIGS. 6 and 7 is a preferred embodiment, because the entire tip can be made in a single processing operation. Furthermore the handling of the syringe is easier because the retaining section is omitted during use, along with a reduction of the danger of injury.

FIGS. 8 and 9 show an embodiment of the syringe according to the invention similar to that of FIGS. 6 and 7. FIG. 8 shows the stored syringe and FIG. 9 shows the syringe in use.

The syringe of FIGS. 8 and 9 also has a sterilization filter 12, which allows sterile aeration of the container 10 during use. When a liquid is fed into this container, the air displaced must be discharged, or, when liquid is drawn from this container, air must flow in, which should be sterile.

For connection of a sterile filter the hollow peg 9 of the syringe has an extended hollow cylindrical section 9c and a connector 9d formed at the so-called syringe head, which connects to an interior passage in the hollow peg 9 and is equipped with the sterile filter.

The sterile filter can also be releasably integrated in the hollow peg in other embodiments.

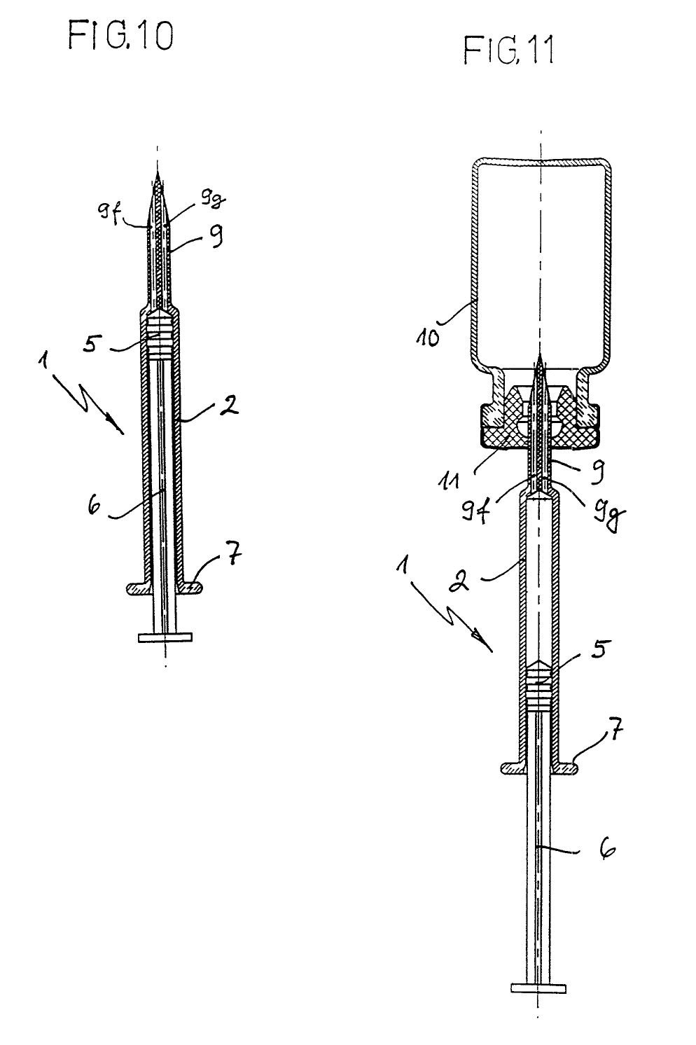

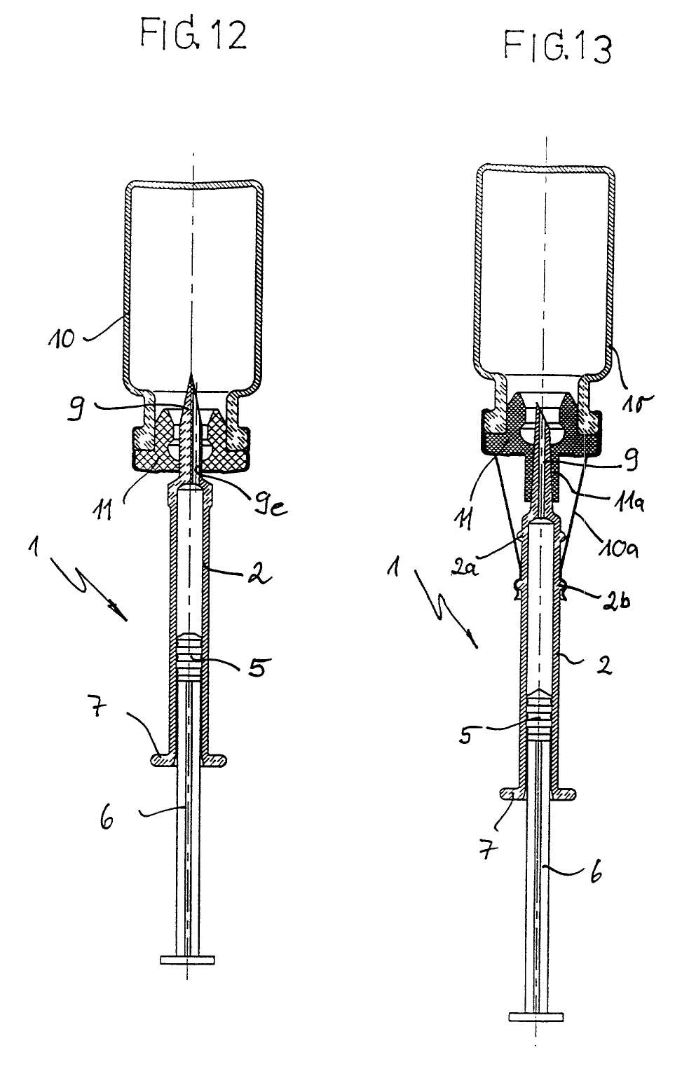

All embodiments of the syringe according to the invention shown up to now have a hollow peg 9 with a through-going bevel on its front end. In contrast, in the embodiment of the syringe shown in FIGS. 10 to 12 the hollow peg is conical, comes to a point at its penetrating end and has lateral openings in its conical surface. In the embodiment according to FIG. 12 the hollow peg 9 is provided with only a single eccentrically positioned flow passage 9e. In the embodiment of FIGS. 10 and 11 the hollow peg 9 has two flow passages 9f and 9g that are symmetrically located with respect to the syringe longitudinal axis.

The structure of the hollow peg 9 coming to a tip, also is called a lancet, is better suited for penetration of the closure of some containers than the beveled structure according to FIGS. 1 to 9 with the asymmetric tip.

FIG. 13 shows a container 10 with a special closure 11 having a connector 11a and a holder 10a for the syringe in the embodiment of FIG. 6, which has bulging projections 2a and 2b for a shape- or form-locking connection with a complementary end of the holder 10a.

This special embodiment according to FIG. 13 permits storage of the syringe 1 together with the preparation container 10. While being stored the cone-shaped holder 10 is fixed on a bulging projection 2a (closest to the head end of the syringe). In this position the elastomeric closure 11 of the container is not pierced.

The hollow peg 9 is only sealed in the molded connector 11a. In this pre-assembled syringe and container arrangement the syringe 1 and the medication container 10 are thus closed in the storage state. Next shortly prior to use the syringe is pushed into the position shown in FIG. 13, so that the holder 10a engages on the bulging projection 2b furthest from the head end. In this position now the contents of the syringe 1 can be transferred into the bottle 10. After drawing the mixed contents up into the syringe from the bottle it can now be withdrawn from the connector 11a and the holder 10a and can now, as already described, be again used. The pre-positioning of the syringe on the bottle facilitates prevention of use by non-professionals.

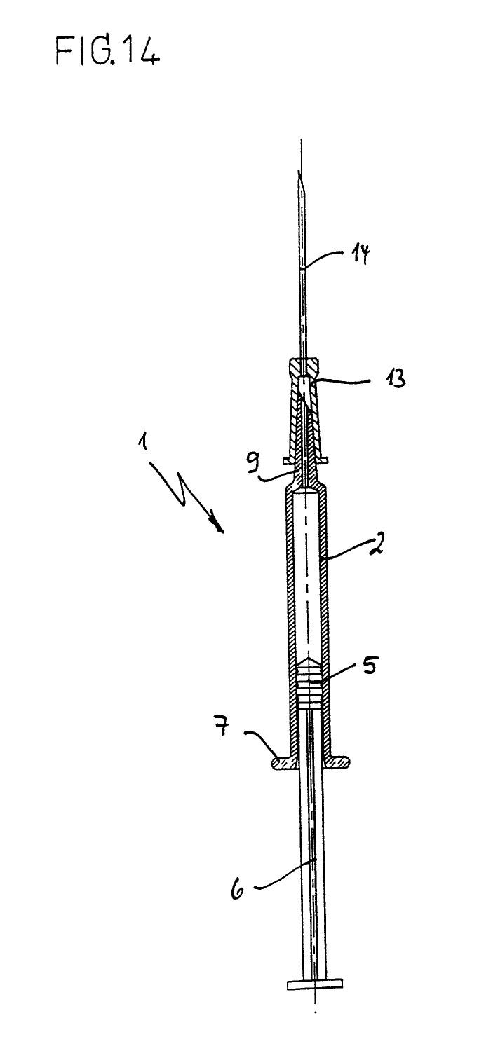

The syringe of FIG. 6 is shown in FIG. 14. A standard hollow needle 14 is mounted on the hollow peg 9 by means of a needle carrier 13, which guarantees that the syringe according to the invention is universally usable.

Typically the cross-section of the hollow peg is round. However it can also be oval.

The hollow peg 9 is centrally mounted on the injector or syringe body 1 in FIGS. 1 to 14. It is also conceivable to mount the hollow peg off center.

The syringes of FIGS. 1 to 14 can also be formed as finished products. They are then filled, for example with a solvent.

The manufacture of the syringe according to FIGS. 1 to 14, the choice of plastic material, optionally the application of a barrier layer or lubricating layer and the filling of the injector occur according to the state of the art.

The disclosure in German Patent Application 199 27 201.8-41 of Jun. 15, 1999 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a syringe or injector device for medical purposes, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A syringe for transferring a medical substance from a medical container provided with an elastomeric closure into an infusion container, said syringe comprising
a plastic syringe body (1) having a front end; and
a plastic hollow peg (9) arranged on the front end of the plastic syringe body (1), wherein said plastic hollow peg (9) includes means for piercing the elastomeric closure (11) of the infusion container (10).

2. The syringe as defined in claim 1, wherein said plastic syringe body (1) has a conical-shaped syringe head (3) and said plastic hollow peg includes a retaining section (8) formed for engagement with said syringe head (3).

3. The syringe as defined in claim 1, wherein said plastic hollow peg (9) is in one-piece with said plastic syringe body (1) and forms an outlet end potion of said plastic syringe body (1).

4. The syringe as defined in claim 1 or 3, wherein said hollow peg (9) is cylindrical.

5. The syringe as defined in claim 1 or 3, wherein said hollow peg (9) is conical and tapered.

6. The syringe as defined in claim 1 or 3, wherein said hollow peg (9) has a bevel (9a) extending over an entire diameter of said hollow peg at a piercing end of said hollow peg (9).

7. The syringe as defined in claim 1, wherein said hollow peg (9) is conical and comes to a point.

8. The syringe as defined in claim 7, wherein said hollow peg (9) is provided with an eccentrically located flow passage (9e).

9. The syringe as defined in claim 7, wherein said hollow peg (9) is provided with two symmetrically arranged flow passages (9f, 9g).

10. The syringe as defined in claim 1, further comprising a sterilization filter (12) mounted on a body end (9c) of the hollow peg (9).

11. The syringe as defined in claim 10, further comprising a connector (9d) for connecting said sterilization filter (12) to the hollow peg (9).

12. The syringe as defined in claim 1, wherein said hollow peg (9) has an oval cross-section.

13. The syringe as defined in claim 1, wherein said hollow peg (9) is mounted eccentrically on said syringe body (1).

14. The syringe as defined in claim 1, wherein said hollow peg (9) is formed so that a standard hollow needle can be placed on said hollow peg (9).

15. A pre-assembled syringe and container arrangement comprising a syringe for transferring a medical substance from a medical container (10) into an infusion container and the medical container, wherein said syringe comprising a plastic syringe body (1) and a plastic hollow peg (9) arranged at the front end of the plastic syringe body (1), said medical container (10) has an elastomeric closure (11) and said plastic hollow peg (9) includes means for penetrating said elastomeric closure, whereby said medical substance may be drawn from said medical container (10) by said syringe.

16. A syringe and container arrangement comprising
a syringe for transferring a medical substance from a preparation container (10) into an infusion container, said syringe comprising a plastic syringe body (1) having at least two bulging projections (2a,2b) spaced axially from each other on an exterior surface thereof and having a front end and a plastic hollow peg (9) arranged on the front end of the plastic syringe body (1);
the preparation container (10) for holding the medical substance to be transferred to the infusion container, said preparation container having an elastomeric closure (11), said elastomeric closure (11) including a connector (11a) for engaging a front portion of the hollow peg (9) to seal a connection thus formed between the preparation container (10) and the syringe (1) in a storage condition and said hollow peg including means for penetrating said elastomeric closure to draw the medical substance from the preparation container (10); and
a holder (10a) for holding said syringe body (1) on said preparation container (10), wherein said holder comprises means for engagement of said elastomeric closure (11) with one of said at least two bulging projections (2a,2b) of said syringe (1) in said storage condition, whereby the syringe is closed by the connection with the connector (11a) but the hollow peg (9) does not penetrate the elastomeric closure, or alternatively with another of said at least two bulging projections so that said elastomeric closure (11) is penetrated by said hollow peg (9).

17. The syringe and container arrangement as defined in claim 16, wherein said syringe is pre-filled with another substance for delivery to said preparation container.
